(19) United States
(12) Patent Application Publication
(10) Pub. No.: US 2010/0113927 A1
(43) Pub. Date: May 6, 2010

(54) ULTRASONIC APPARATUS

(76) Inventors: Mariko YAMAMOTO, Kokubunji (JP); Shin-ichiro Umemura, Sendai (JP); Takashi Azuma, Kodiara (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/545,987

(22) Filed: Aug. 24, 2009

(30) Foreign Application Priority Data
Oct. 30, 2008 (JP) 2008-279986

(51) Int. Cl. A61B 8/00

(52) U.S. Cl. 600/439

(57) ABSTRACT

A high-quality image pickup is performed even when there is a strong reflector, and image pickup and therapy are performed without reducing overall sound pressure even when there is a site which should not be exposed to a high sound pressure. Data for setting a desired beam is acquired, the position and intensity of a site to be avoided are detected from the data, the position and intensity are converted into a desired beam shape, focus data to form a beam along the desired beam shape is calculated, and the focus data is used to perform image generation or treatment.

### BACKGROUND OF THE INVENTION

1. Field of the Invention

The present invention relates to an ultrasonic apparatus, and in particular to an ultrasonic apparatus suitable for medical uses.

2. Background Art

In medical ultrasonic apparatuses, realization of high quality image and attainment of safety in medical practice are crucial issues. In particular, significant challenges are to improve a problem that a strong reflector, if present, acts as a noise source often leading to a decline of image quality in which a real image is superimposed with a false image, and a problem that during a high intensity focused ultrasound (HIFU) therapy, when there is a portion which should not be exposed to a strong sound pressure, the overall sound pressure needs to be decreased resulting in decreases of the sound pressure and the range of cautery site and an increase of cautery time.

Examples of strong reflectors include a rib, a diaphragm, a metallic probe in an HIFU device, etc. A decline of image quality due to a strong reflector is common occurrence; there are practiced, as the method to solve such a problem, a manual method which eliminates fault images by determining them exploiting the fact that changing the probe position will result in a change in the relative positions of a real image and a fault image, or averaging them by changing the probe position and time, and a technical method which eliminates them by averaging processing such as a compound method. Patent Document 1 discloses a method for recovering image quality so that the structure of a living body can be determined in the entire image by locally adjusting the luminance through image processing when the position of a strong reflector is known in advance.

[Patent Document 1] JP Patent Publication (Kokai) No. 2000-37393

### SUMMARY OF THE INVENTION

However, in either of the above described conventional methods, the signal to noise ratio has not been improved, and in the commonly practiced manual and technical averaging methods, a further problem arises in that time resolution declines. Moreover, there is no method disclosed which enables to reduce the sound pressure only at a specified position.

The objects of the present invention are to perform a high-quality image pickup by improving the signal-to-noise ratio without reducing time resolution even when a strong reflector is present, and to provide an ultrasonic apparatus which enables to perform an image pickup and treatment without reducing the overall sound pressure even when there is a site which should not be exposed to strong sound pressure.

The present invention intends to realize high quality image and to ensure safety by means of sound field design. The ultrasonic apparatus of the present invention comprises: a probe including a plurality of elements for transmitting or receiving ultrasound; a transmission beamformer for imparting directivity to an ultrasonic signal upon transmission to a subject by the plurality of elements; a reception beamformer for summing each ultrasonic signal received by the plurality of elements, along with directivity thereof; a signal processing part for signal-processing and imaging the signal outputted by the reception beamformer; and display means for displaying the image outputted by the signal processing part.

The signal processing part includes a desired-beam-shape setting part for setting a desired beam shape, and a focus data generation part which receives a desired beam shape as input and calculates focus data to generate a beam along the desired beam shape. At least one of the transmission beamformer and the reception beamformer generates a beam by using the focus data outputted by the focus data generation part. Alternatively, receive signals for every element of the probe are stored in a memory and the focus data outputted by the focus data generation part is applied to the receive signals to reconfigure an image.

According to the ultrasonic apparatus of the present invention, it is possible to form a sound field in which the sound pressure at an intended position is suppressed, and to improve the signal-to-noise ratio without reducing the time resolution even when there is a strong reflector which acts as a noise source, enabling to obtain a high-quality image. Further, it is possible to perform image pickup and treatments without reducing the overall sound pressure even when there is a site which should not be exposed to a high sound pressure.

### BRIEF DESCRIPTION OF THE DRAWINGS

FIG. 1A is a block diagram to show a configuration example of an ultrasonic apparatus of the present invention.

FIG. 1B is a flowchart to illustrate a flow of processing.

FIG. 2 is a flowchart to illustrate the processing of a signal processing part.

FIG. 3 is a flowchart to illustrate the processing of a desired-beam-shape setting part.

FIG. 4A is a flowchart to illustrate the processing of a focus data generation part.

FIG. 4B is an explanatory diagram of a function W and an eigenfunction φ.

FIG. 5A is a conceptual diagram to illustrate an example of the processing to acquire data for determining a desired sound field.

FIG. 5B is a conceptual diagram to illustrate an example of the processing to set a desired beam shape.

FIG. 5C is a conceptual diagram to illustrate an example of the processing to generate an image.

FIG. 6 illustrates an example of the numerical relationship between the position and intensity of a site to be avoided and a desired beam shape.

FIG. 7 shows a calculation example of the input and output of a focus data generation part.

FIG. 8 is a simulation image to validate the effect of an ultrasonic apparatus of the present invention.

FIG. 9A is a flowchart to illustrate the flow of the processing which calculates focus data from a desired-sound-field determination data by real time processing and uses the focus data.

FIG. 9B is a flowchart to illustrate the flow of the processing which calculates focus data from a desired-sound-field determination data by offline processing and uses the focus data.

FIG. 10 shows an example of the processing of a desired-beam-shape setting part.

FIG. 11 is a flowchart to illustrate an example of the processing of a focus data generation part.

FIG. 12 shows a sound field which is formed by a desired beam shape and calculated focus data in the case in which the spatial dimension of W of the focus data generation part of the present invention is two-dimensional.

FIG. 13 shows a calculated connection pattern and focus data, and a sound field formed of those in the case of a discretizing operation G in the focus data generation part of the present invention.

### DETAILED DESCRIPTION OF THE PREFERRED EMBODIMENTS

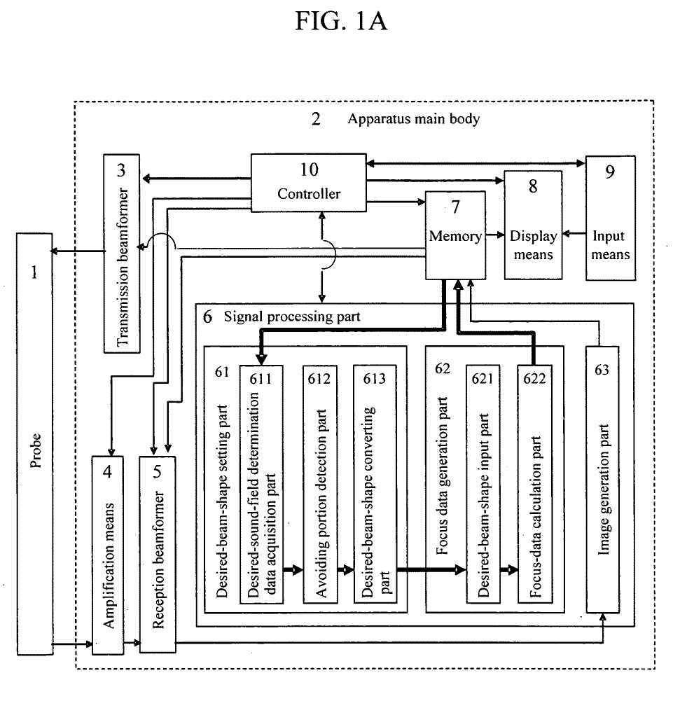

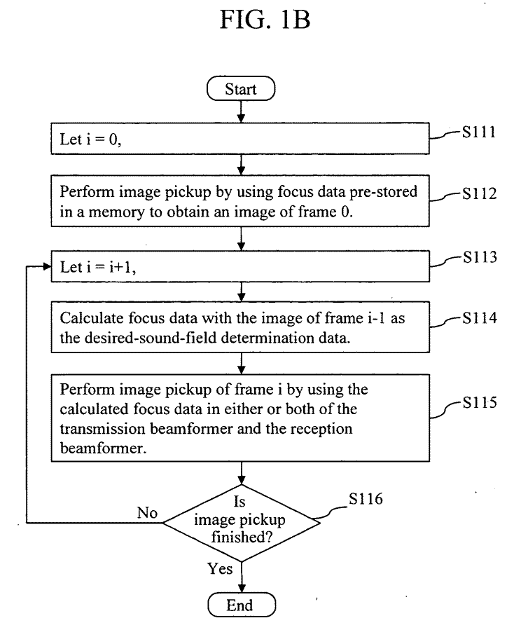

First, a first embodiment of the present invention will be described with reference to FIGS. 1A to 9.

FIG. 1A is a block diagram to show a configuration example of the ultrasonic apparatus of the first embodiment of the present invention. A probe 1 comprises a plurality of elements. The apparatus main body 2 includes a transmission beamformer 3, amplification means 4, a reception beamformer 5, a signal processing part 6, a memory 7, display means 8, input means 9, and a controller 10. The signal processing part 6 includes a desired-beam-shape setting part 61, a focus data generation part 62, and an image generation part 63. Further, the desired-beam-shape setting part 61 includes a desired-sound-field determination data acquisition part 611, an avoiding portion detection part 612, and a desired-beam-shape converting part 613, and the focus data generation part 62 includes a desired-beam-shape input part 621, and a focus-data calculation part 622.

An ultrasonic pulse generated at the transmission beamformer 3 is transmitted from the probe 1 into a living body and the ultrasound reflected from the living body is received by the probe 1. The receive signal is inputted into the amplification means 4 to be amplified and the reception beamformer 5 performs phased summation thereof The receive signal outputted by the reception beamformer 5 is inputted into the image generation part 63 of the signal processing part 6 and imaging thereof is performed. The created image is stored in the memory 7 and thereafter is read out and interpolated to be displayed on the display means 8. It is noted that these processing is controlled by the controller 10.

The focus data to be used for the beamforming in the transmission beamformer 3 and the reception beamformer 5 is stored in the memory 7 in advance and may be read out from the memory 7 upon image pickup. Alternatively, it may be calculated from the desired-sound-field determination data which is the data stored in the memory 7. Here, the focus data refers to time delays and pulse intensities, or complex amplitudes in numerical expression, which are given to a plurality of elements to impart directivity to an ultrasonic signal upon transmission/reception thereof. The desired-sound-field determination data may be, for example, the result of performing the above described image pickup, which is temporarily stored in the memory 7. This desired-sound-field determination data is more easily understood when regarding it as a pre-scan result. When calculating the focus data from the desired-sound-field determination data, assuming that the desired-sound-field determination data is temporarily stored in the memory 7, and that the calculated focus data is also temporarily stored in the memory 7, the flow of the signal will be as shown by thick arrow lines in FIG. 1A. Specifically, the flow is such that desired-beam-shape setting part 61 reads out the desired-sound-field determination data from the memory 7, converts it into a desired beam shape, and outputs it to the focus data generation part 62, and the focus data generation part 62 converts the inputted desired beam shape into focus data and outputs it to the memory 7.

FIG. 1B is a flowchart to illustrate the flow of the processing to calculate focus data from a desired-sound-field determination data and to use the focus data.

First, upon the start of image pickup (START), an integer i, which represents a frame number, is set to be 0 (S111), and image pickup is performed by using focus data pre-stored in a memory to obtain an image of frame 0 (S112). Next, unless the image pickup is ended such as a result of the power source being shut down (S116), the processing is continued in such a way that i is set to be i+1 (S113), focus data is calculated with the image of frame i−1 as the desired-sound-field determination data (S114), and image pickup of frame i is performed by using the calculated focus data in either or both of the transmission beamformer and the reception beamformer (S115).

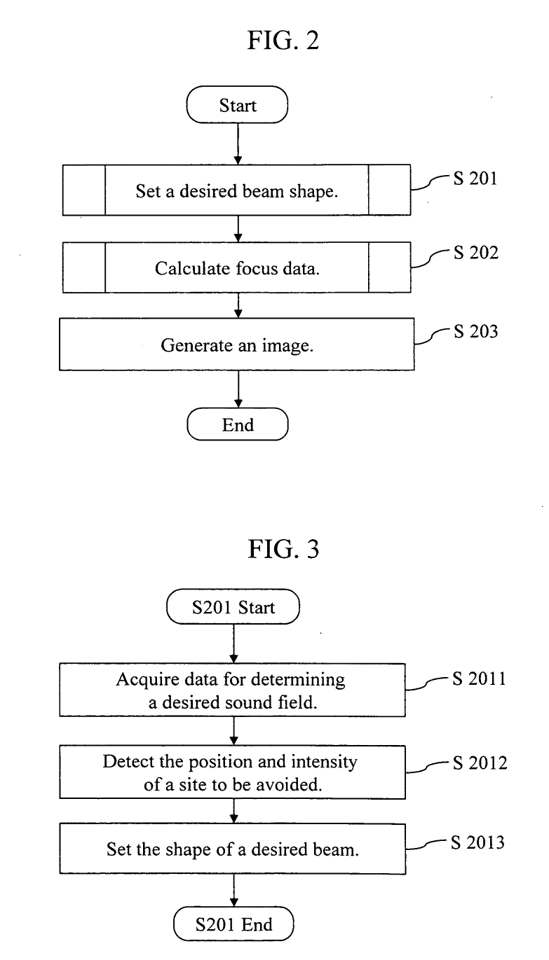

Hereafter, detailed description will be made with reference to FIGS. 2 to 8. First, the processing of the signal processing part 6 will be described using the flowcharts of FIGS. 2 to 4A.

FIG. 2 is a flowchart to illustrate the processing of the signal processing part 6. Upon the activation of the signal processing part 6 (start), the desired-beam-shape setting part 61 sets a desired beam shape (S201), the focus data generation part 62 calculates focus data for generating a beam having a shape close to the desired beam shape (S202), and with ultrasound being transmitted/received to and from an imaging target using the calculated focus data, received data is input to the image generation part 63 to generate an image (S203).

FIG. 3 is a flowchart to illustrate the processing to set the shape of a desired beam (S201) among the processing in the signal processing part 6. Upon the start of the processing (S201) to set a desired beam shape (S201 START), the desired-sound-field determination data acquisition part 611 acquires data for determining a desired sound field (S2011), the avoiding portion detection part 612 detects the position and intensity of the site to be avoided (S2012), and the desired-beam-shape converting part 613 converts the position and intensity of the site to be avoided into the shape of a desired beam to set a desired beam shape (S2013).

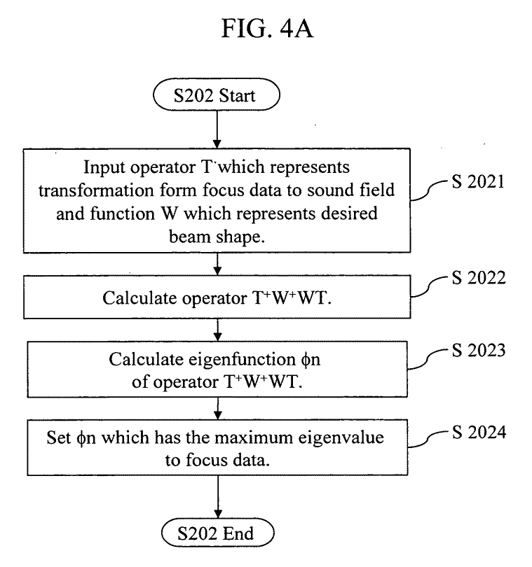

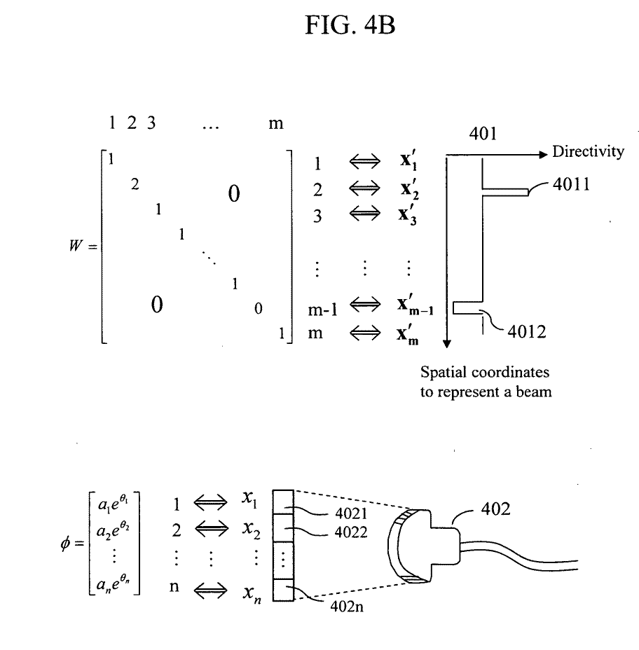

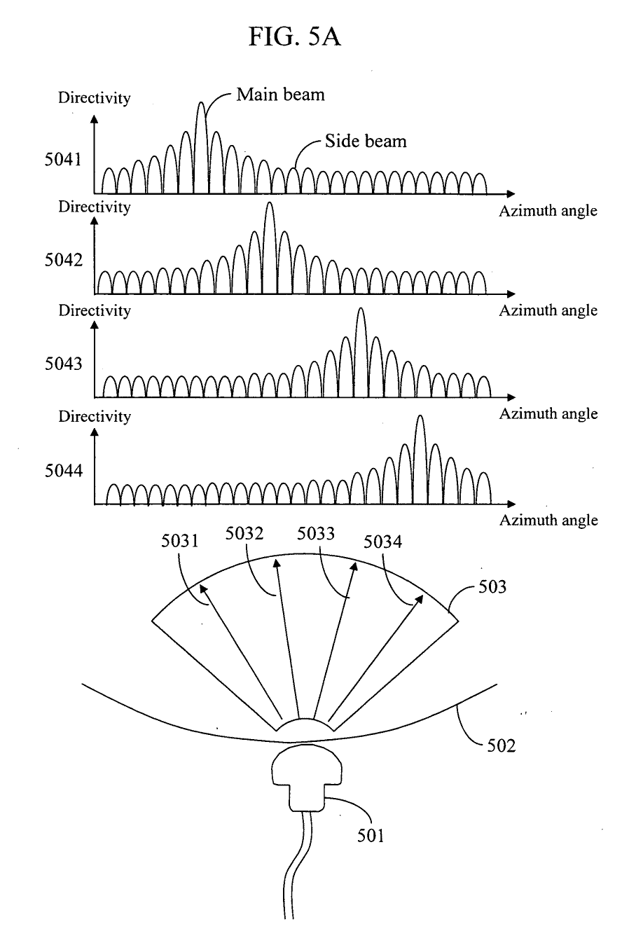

FIG. 4A is a flowchart to illustrate an example of the processing of the focus data generation part 62 (S202) among the processing in the signal processing part 6. Upon the start of the focus data generation part 62 (S202 START), an operator T which represents a transformation from focus data to sound field and a function W which represents a desired beam shape are inputted (S2021), operator T⁺W⁺WT is calculated (S2022), an eigenfunction φn of the operator T⁺W⁺WT is calculated (S2023), and φn which has the maximum eigenvalue is set to focus data (S2024). It is noted that all the operators may be either discrete (a matrix expression or tensor expression) or continuous (a functional expression).

Hereafter, examples of the operator T which represents the transformation from focus data to sound field, the function W which represents a desired beam shape, and the eigenfunction φn of the operator T⁺W⁺WT in FIG. 4A are shown. In order to avoid any confusion in subscripts, herein the eigenfunction φn is denoted by φ.

$$T = {[ \begin{matrix}\frac{{e}^{i k \sqrt{{z}^{2} + {( {x}_{1}^{′} - {x}_{1} )}^{2}}}}{\sqrt{{z}^{2} + {( {x}_{1}^{′} - {x}_{1} )}^{2}}} & \frac{{e}^{i k \sqrt{{z}^{2} + {( {x}_{1}^{′} - {x}_{2} )}^{2}}}}{\sqrt{{z}^{2} + {( {x}_{1}^{′} - {x}_{2} )}^{2}}} & & \frac{{e}^{i k \sqrt{{z}^{2} + {( {x}_{1}^{′} - {x}_{n} )}^{2}}}}{\sqrt{{z}^{2} + {( {x}_{1}^{′} - {x}_{n} )}^{2}}} \\ \frac{{e}^{i k \sqrt{{z}^{2} + {( {x}_{2}^{′} - {x}_{1} )}^{2}}}}{\sqrt{{z}^{2} + {( {x}_{2}^{′} - {x}_{1} )}^{2}}} & \frac{{e}^{i k \sqrt{{z}^{2} + {( {x}_{2}^{′} - {x}_{2} )}^{2}}}}{\sqrt{{z}^{2} + {( {x}_{2}^{′} - {x}_{2} )}^{2}}} & & ⋮ \\ ⋮ & & ⋱ & \\ \frac{{e}^{i k \sqrt{{z}^{2} + {( {x}_{m}^{′} - {x}_{1} )}^{2}}}}{\sqrt{{z}^{2} + {( {x}_{m}^{′} - {x}_{1} )}^{2}}} & … & & \frac{{e}^{i k \sqrt{{z}^{2} + {( {x}_{n}^{′} - {x}_{n} )}^{2}}}}{\sqrt{{z}^{2} + {( {x}_{n}^{′} - {x}_{n} )}^{2}}}\end{matrix} ]}^{\begin{matrix}\begin{matrix}\begin{matrix}1 & & & \end{matrix} & & & & & 2 & & & & \end{matrix} & … & & & & n\end{matrix}} \begin{matrix}\begin{matrix}1 \\ \\ \\ 2 \\ \end{matrix} \\ ⋮ \\ \\ m\end{matrix}$$

$$W = {[ \begin{matrix}1 & & & & & & & \\ & 2 & & & & & & \\ & & 1 & & & 0 & & \\ & & & 1 & & & & \\ & & & & ⋱ & & & \\ & & & & & 1 & & \\ & & 0 & & & & 0 & \\ & & & & & & & 1\end{matrix} ]}^{\begin{matrix}1 & 2 & 3 & & … & & & m\end{matrix}} \begin{matrix}1 \\ 2 \\ 3 \\ \\ ⋮ \\ \\ \\ m\end{matrix}$$

$$φ = [ \begin{matrix}{a}_{1} {e}^{{θ}_{1}} \\ {a}_{2} {e}^{{θ}_{2}} \\ ⋮ \\ {a}_{n} {e}^{{θ}_{n}}\end{matrix} ]$$

In the present example, it is supposed that all the operators be discrete operators, the space for representing a beam shape be one dimensional, the array of the oscillation elements of the probe 1 be one dimensional, the discretization number of the space for representing the beam shape be m, the number of the oscillation elements be n, the position coordinates of the space for representing the beam shape be x′₁=(x′₁, z)˜x′ₘ=(x′ₘ, z), and the position coordinates of the oscillation element be (x₁, 0)˜(xₙ, 0). In this case, the operator T which represents a transformation from focus data to sound field will be a matrix with m rows and n columns. The element at the i-th row and the j-th column of the matrix T indicates the intensity of the sound field which is formed by the j-th oscillation element in the i-th spatial position. It is noted that the operator T in the present example will be a matrix representing a Fourier transform at a limit of x′₁/z→0, which is equivalent to that the directivity of beam becomes a Fourier transform of the focus data to be provided to the oscillation element by paraxial approximation. The function W for representing the desired beam shape will be a diagonal matrix with m rows and n columns. When the desired beam shape is for example a shape 401 shown in FIG. 4B, specifically, a shape with a main beam 4011 at a coordinate (x′₂, z) and a suppressed portion 4012 at a coordinate (x′ₘ₋₁, z), the diagonal elements will be a matrix in which the element at the x′₂-th row and the x′₂-th column has a large value, for example, 2, the element at the x′ₘ₋₁-th row and the x′ₘ₋₁-th column has a small value, for example, 0 and other elements have intermediate values thereof, for example, 1. In such a case, the eigenfunction φ of the operator T⁺W⁺WT will a complex vector with n rows. Each element, which is generally a complex number, corresponds to the focus data of the oscillation elements 4021 to 402n of the probe 402. Specifically, the amplitude aᵢ and the phase θᵢ of the i-th element correspond to the acoustic intensity and (delay time)*(sound speed) when the oscillation element 402i performs beamforming.

By using the below described equations, the arrangement by which focus data is determined as the eigenfunction φ of the operator T⁺W⁺WT when generating a beam having a shape W will be described.

$$\begin{matrix}B = T φ & ( 1 ) \\ \begin{matrix}J = {‖ WB ‖}^{2} - λ {‖ φ ‖}^{2} \\ = {( WT φ )}^{+} ( WT φ ) - λ {φ}^{+} φ \\ = {φ}^{+} {T}^{+} {W}^{+} WT φ - λ {φ}^{+} φ\end{matrix} & ( 2 ) \\ δ J = δ {φ}^{+} \cdot ( {T}^{+} {W}^{+} WT φ - λ φ ) & ( 3 ) \\ \begin{matrix}δ J = 0 \\ ⇔ {T}^{+} {W}^{+} WT φ - λ φ = 0 \\ ⇔ {T}^{+} {W}^{+} WT φ = λ φ\end{matrix} & ( 4 )\end{matrix}$$

Suppose a beam is created with focus data φ. In this case, since the operator to represent the transformation from focus data to sound field is represented by T, the shape of beam is given as B shown in Equation (1). Here, the degree of coincidence between the beam shape B created by the focus data φ and the desired beam shape W is represented by a square ∥WB∥² of the product of the operators, WB. This will be well understood when considering that the operators W and T are matrices, and the degree of coincidence of two vectors is represented by the square of the scalar product between the vectors. On the other hand, the sum of the sound pressure outputted from the probe is represented by the square ∥φ∥² of the norm of focus data φ. It can be regarded that the sum of the sound pressure outputted from the probe approximately represents the acoustic intensity at a focus point.

Here, suppose that it is the object to determine the focus data for making a beam which has a shape coinciding as much as possible with the desired beam shape W under the condition of a constant acoustic intensity at the focus point. Then, formulating this problem by the calculus of variations, the first line of Equation (2) is obtained as an evaluation function. Where λ is an undetermined coefficient of Lagrange, and the term to which the coefficient is applied is a constraint. By substituting Equation (1) into the evaluation function (2) and rearranging it, the first line of the evaluation function (2) is rearranged as the third line of Equation (2), and taking variations thereof will result in Equation (3). Since a minute change δφ⁺ is arbitrary, it is seen that by deforming the first line of the conditional equation (4), which indicates that variations is 0, the problem results in an eigenvalue problem relating to φ as obtained in the third line of Equation (4). That is, determining the eigenfunction φ of the operator T⁺W⁺WT and using it as the focus data will result in the generation of a beam which has a shape as close as possible to the shape W under the condition of a constant acoustic intensity at a focus point.

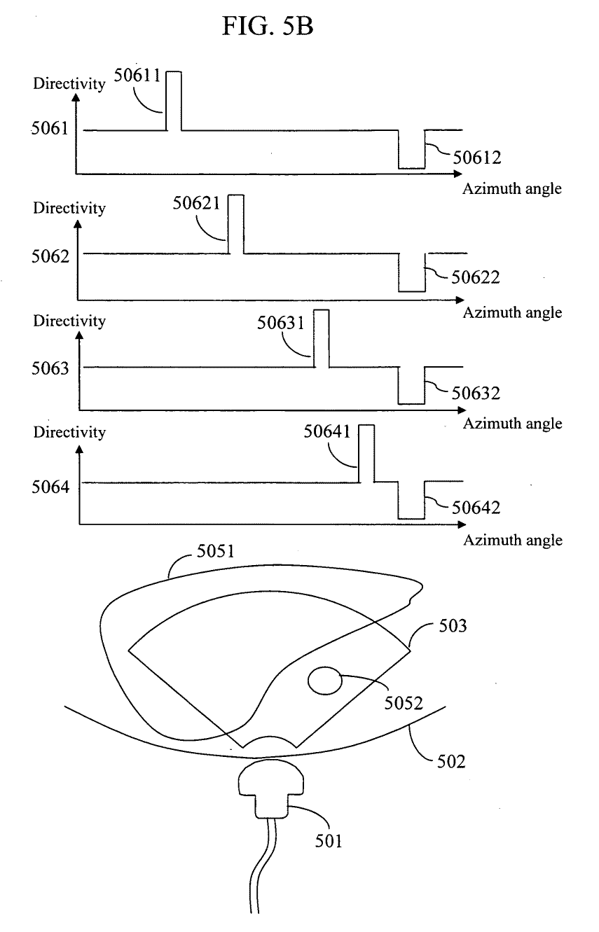

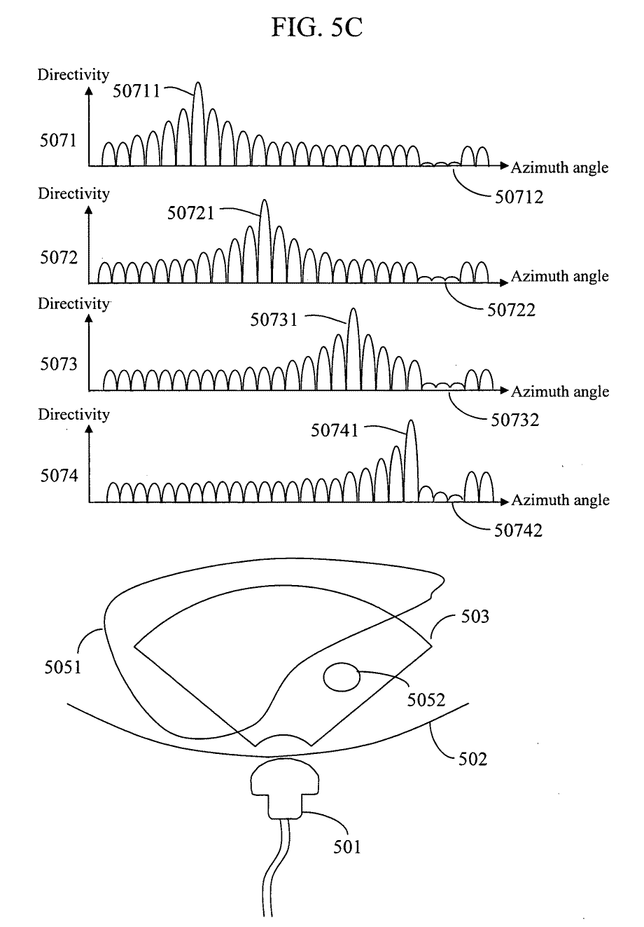

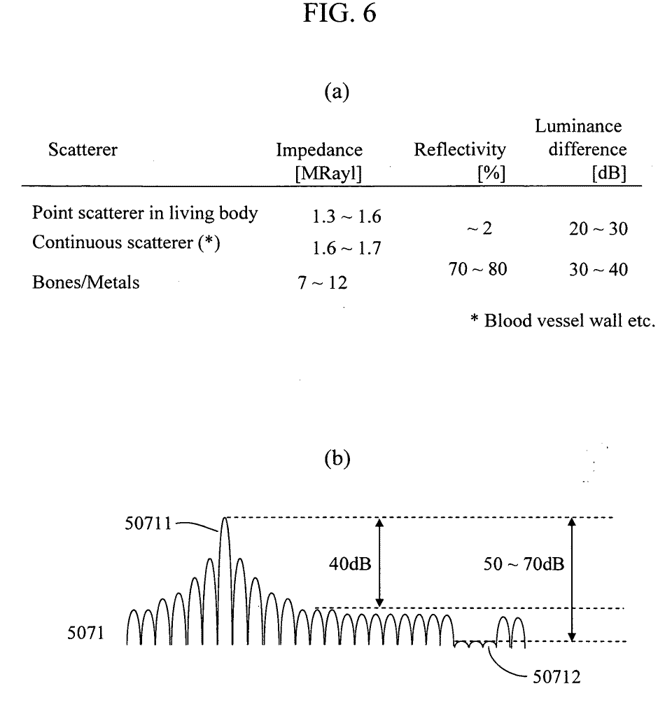

Hereafter, the processing procedure of the signal processing part 6 will be described with reference to the conceptual diagrams of FIGS. 5A to 5C and the explanatory diagram of FIG. 6. FIGS. 5A to 5C are conceptual diagrams to illustrate an example of the image pickup procedure by an ultrasonic apparatus of the present invention.

FIG. 5A is a conceptual diagram to illustrate an example of the processing (S2011) to acquire data for determining a desired sound field. A numeral 501 denotes a probe, 502 a subject such as a human body, 503 an image pickup region, 5031 to 5034 deflection directions of beams in one transmission/reception respectively, and 5041 to 5044 directivities of beams when transmitting or receiving ultrasound in deflection directions 5031 to 5034 of the beams. The horizontal axis represents azimuth angle and the vertical axis represents directivity, and a main beam and side beams are included in the present figure.

Upon the start of the processing (S2011) to acquire data for determining a desired sound field, for example, the desired-sound-field determination data acquisition part 611 sets deflection directions 5031 to 5034 into the image pickup region 503 from the probe 501, and generates beams having directivities 5041 to 5044 for respective deflection directions to acquire ultrasonic signals and turn them into images to be provided as desired-sound-field determination data. It is noted that in the present example, although an example of transmitting/receiving ultrasonic sound is given for the sake of clarity of description, the desired-sound-field determination data acquisition part 611 may temporarily store the image pickup result of the frame preceding by one frame in a memory as in an example given in FIG. 1B and read out the image pickup result of the frame preceding by one frame from the memory to be provided as desired-sound-field determination data.

FIG. 5B is a conceptual diagram to illustrate an example of the processing (S2012 and S2013) to detect the position and intensity of a site to be avoided and to set a desired beam shape. The image in the image pickup region 503 is the desired-sound-field determination data, and 5051 in the image is a subject to be imaged, and 5052 is a strong reflector such as a rib which acts as a noise source for image pickup of the subject. Numerals 5061 to 5064 represent desired beam shapes when transmitting/receiving ultrasound in deflection directions 5031 to 5034 in FIG. 5A. To be specific, numerals 50611 to 50641 represent regions called a main beam where sound pressure is large reflecting the deflection directions, and numerals 50612 to 50642 represent suppressed portions of sound pressure reflecting the position of the strong reflector 5052.

Upon the start of the processing (S2012) to detect the position and intensity of a site to be avoided, an avoiding portion detection part 612 detects the position and intensity of the strong reflector 5052, which acts as a noise source, from the desired-sound-field determination data. Next, a desired-beam-shape converting part 613 converts the position and intensity of the site to be avoided into a desired beam shape and sets desired beam shapes 5061 to 5064 which include main beams 50611 to 50641 of which positions change with the deflection directions 5031 to 5034, and suppressed regions 50612 to 50642 of which positions do not change with the deflection directions 5031 to 5034 (S2012).

It is noted that the above described processing (S2012 and S2013) may either be manually set by a user or be automatically set by the internal processing of the apparatus. To be specific, arrangement may be made such that display means 8 displays desired-sound-field determination data, and the user visually recognizes the position and intensity of the strong reflector 5052 and determines a desired beam shape based on the user's knowledge to manually input it by using input means 9. Alternatively, a signal processing part 6 may read desired-sound-field determination data and a signal processing part may perform image processing such as pattern recognition to automatically detect the position and intensity of the strong reflector 5052 and automatically determine and set desired beam shapes 5061 to 5064 for suppressing the influence of the site to be avoided.

FIG. 5C is a conceptual diagram to illustrate an example of the processing (S203) in which an image generation part 63 generates an image. Numerals 5071 to 5074 represent the directivities of beams when transmitting or receiving ultrasound in deflection directions 5031 to 5034.

Upon the start of the processing (S203) of generating an image, a focus data generation part 62, which receives desired beam shapes 5061 to 5064 as input to calculate focus data, inputs the focus data into a transmission beamformer 3 and/or a reception beamformer 5 and transmits/receives an ultrasonic beam which has the directivities 5071 to 5074 and includes main beams 50711 to 50741 having a large sound pressure in the deflection directions, and suppressed portions 50712 to 50742 in the direction of the strong reflector 5052, to and from a subject through a probe 1 thereby generating an image.

FIG. 6 illustrates an example of the numerical relationship between the position and intensity of the site to be avoided and the beam shape when the desired-beam-shape converting part 613 converts the position and intensity of the site to be avoided into a desired beam shape.

FIG. 6(a) is a table to show the impedance of a scatterer making up a subject of the ultrasonic apparatus, the reflectivity of ultrasound when various scatterers are adjacent to each other, and the luminance difference on an image. As a typical scatterer, a point reflector in a living body, a continuous reflector such as a blood vessel wall, and bones/metals etc. are considered. The impedance of a point reflector in a living body is about 1.3˜1.6 MRayl, that of a continuous reflector such as a blood vessel wall is about 1.6˜1.7 MRayl, and that of bones/metals is about 7˜12 MRayl. Therefore, the reflectivity when a point reflector and a continuous reflector are adjacent to each other is about ˜2%, and the reflectivity when a continuous reflector and bones/metals are adjacent to each other is about 70˜80%. Moreover, the luminance difference on an image will be about 20˜30 dB and about 30˜40 dB respectively.

Therefore, in order to pick up an image of a point reflector in a living body without allowing it to be buried in noise caused by bones and metals, it is satisfactory if the intensity difference in the directivity 5071 of beam between the main beam 50711 and the suppressed portion 50712 upon the transmission/reception of ultrasound in the deflection direction 5031 as shown in FIG. 6(b) is (20˜30 dB)+(30˜40) dB, that is, 50˜70 dB. It is noted that in an average directivity without setting a suppressed portion, the sound pressure difference between the main beam 50711 and the side lobe level (the focusing value of lower directivity side) is about 40 dB.

Applying the above described case to the processing of the present invention for description, when the avoiding portion detection part 612 detects a strong reflector 5052 which is 30 dB higher compared with a continuous reflector from desired-sound-field determination data, if the desired-beam-shape converting part 613 sets desired beam shapes 5061 to 5064 having a sound pressure 50 to 60 dB lower than that of the main beam at the position of a strong reflector, it is possible to pick up an image of the point reflector in the living body without allowing it to be buried in noise caused by the strong reflector.

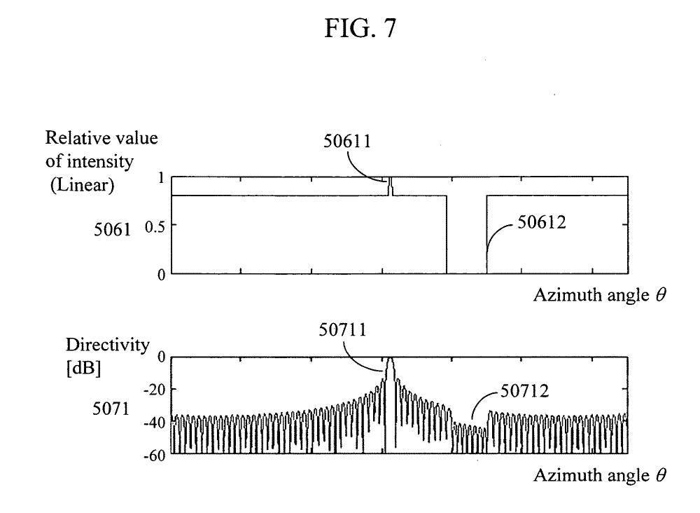

FIG. 7 shows a calculation example of the input and output of a focus data generation part. The upper row is an example of a function W which represents a desired beam shape in the focus data generation part 62, and the lower row is an example of the directivity which is formed by using focus data calculated by the focus data generation part through the processing shown in FIG. 4. The vertical axis of the upper row is represented by linear indication, the vertical axis of the lower row by logarithmic indication, and both horizontal axes by azimuth angle indication. It is seen that only the specified portion 50612 has a significantly lower sound pressure than its surroundings, and the sound pressure difference between the main beam 50711 and the suppressed portion 50712 is about 50 dB. This value indicates a sufficient sound pressure difference for picking up image of a point reflector in a living body without allowing it to be buried in noise caused by bones and metals, as described with reference to FIG. 6. It is noted that in the present example, the operator T which represents transformation from focus data to sound field in the processing S2021 shown in FIG. 4 is a discrete Fourier transformation.

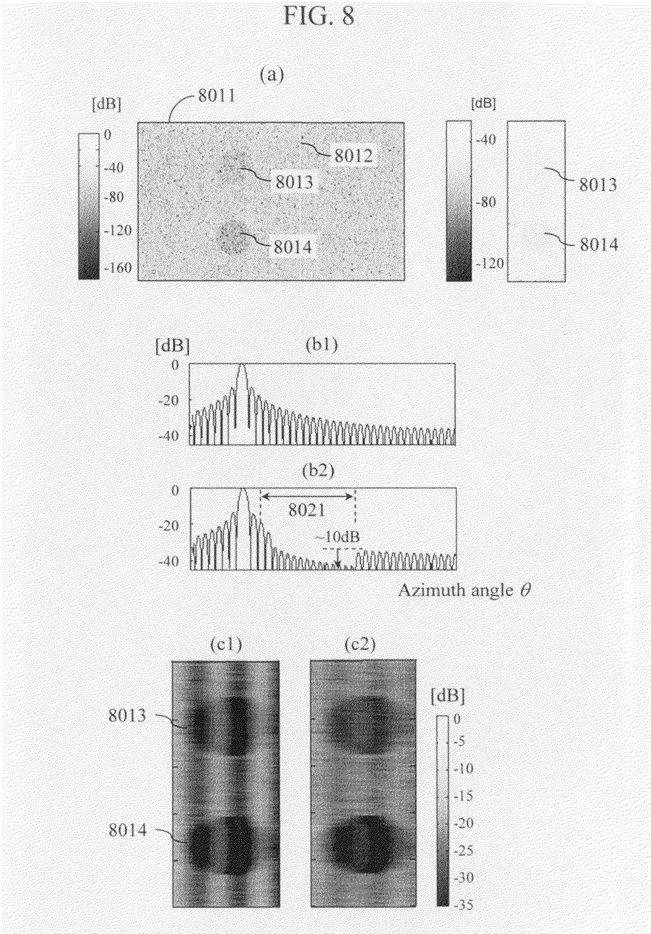

FIG. 8 is a simulation image to verify the effect of an ultrasonic apparatus of the present invention. FIG. 8(a) shows a phantom, FIGS. 8(b1) and 8(b2) show the directivities of the beams used in the simulations according to a conventional method and a method of the present invention, and FIGS. 8(c1) and (c2) show simulation images by a conventional method and a method of the present invention.

The phantom, which is a one-dimensional cyst phantom having a low luminance and includes a thin-plate-shaped strong reflector, includes a strong reflector 8012 of 0 dB and two cysts 8013 and 8014 of −34.0 dB and −40.0 dB in the background 8011 of an average reflectance strength of −30 dB. The diameter of a cyst is about twice the width of the beam. Although the phantom shown in FIG. 8(a) and the beam shown in FIG. 8(b) are assumed to be one-dimensional for the sake of simplicity, the method of the present invention is applicable to 2-dimensional and 3-dimensional image pickup targets and beams. Moreover, to make the cysts 8013 and 8014 of FIG. 8(a) to be more perceivable, an extracted view in which the display range is reduced is placed on the right-hand side. The suppressed portion 8021 for suppressing the influence of a strong reflector within a beam according to the method of the present invention shown in FIG. 8(b2) is configured to be wider in order to ensure such effect, and the sound pressure thereof is adapted to be about 10 dB lower than that of a beam of conventional method in the present example. FIG. 8(a) and FIGS. 8(b1) and (b2) have the same horizontal scale, and FIGS. 8(c1) and (c2) are enlarged views.

It is seen in the simulation image that while fogging caused by a strong reflector is observed in the conventional method shown in FIG. 8(c1), the fogging is suppressed in the method of the present invention shown in FIG. 8(c2). As a result of the fogging being suppressed, perception of shape becomes easy particularly in 8013. It is noted that in the present simulation, since the strong reflector is thin-plate-shaped as shown in 8012, and the beam (b2) is a one-dimensional continuous wave, fogging lying on the cyst has a shape of the side lobe itself, and it is possible to even visually separate and distinguish the luminance change between the fogging which is noise and the circle which is a signal. However, typically, a strong reflector not necessarily has a simple shape and the fogging generally has a complicate shape. As a result of that, the noise due to the fogging having a complicated shape makes it difficult to determine the shape of the structure which is a signal. That is, according to the method of the present invention, in most cases, improvement in visibility of shape exceeding the level shown in FIG. 8(c2) is achieved.

In the method of the present invention, fogging which is noise is suppressed thereby improving the signal-to-noise ratio of signal so that it becomes possible to discriminate a shape 8013 which can not be discriminated by a conventional method. It is noted that an image (c1) according to a conventional method may be used as the desired-sound-field determination data in the present invention. In such a case, the desired-sound-field determination data acquisition part 611 reads desired-sound-field determination data 8011; the avoiding portion detection part 612 detects a strong reflector 8012 which has a luminance 30 dB higher than its surroundings at an azimuth angle 8021; a focus data generation part 62 calculates focus data for making a reception beam (b2) having a suppressed portion 8021 which is 50 dB smaller than the main beam at the azimuth angle at which the strong reflector 8012 is present; and an image generation part 63 generates images. As a result of that, fogging is suppressed and signal-to-noise ratio is improved making it possible to obtain an image (c2) which enables to discriminate shapes 8013 which cannot be discriminated by a conventional method.

According to the configuration as described above, since a signal from a subject is obtained by main beams 50711 to 50741, and noise from a strong reflector 5052 is removed by suppressed portions 50712 to 50742, a high-quality image with a high signal-to-noise ratio can be obtained even when a strong reflector which may be a noise source is present.

To be more specific, according to the configuration as described above, in particular, to the configuration of FIG. 1B, it is possible to calculate focus data in real time from the desired-sound-field determination data and thereby obtain an image in which the influence of noise source is suppressed, and also possible to suppress the influence of the noise source which moves with blood flow and body movement without reducing the frame rate of image.

Moreover, specifically, since noise source is detected by automatic processing, even for a noise source which is not known and unexpected, it is possible to suppress the effect thereof without time and effort.

Further, according to the configuration as described above, considering a region, which should not be exposed to strong sound pressure such as a blood vessel, in place of the site which is supposed to be a strong reflector in the above described example, and a deflection direction in place of a cautery site of HIFU therapy, the sound pressure at the blood vessel part is suppressed and the sound pressure at the cautery site can be increased more than that in a conventional method without damaging the blood vessel, making it possible to perform treatments in a short time while ensuring medical safety. However, in this case, the focus data calculated at step S115 in FIG. 1B is used in the transmission beamformer, or in the transmission beamformer and the reception beamformer.

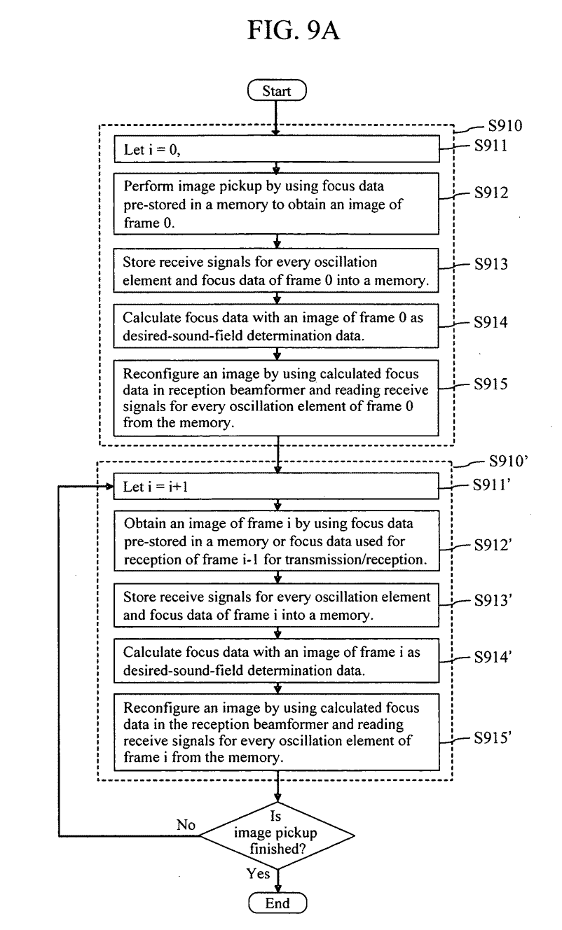

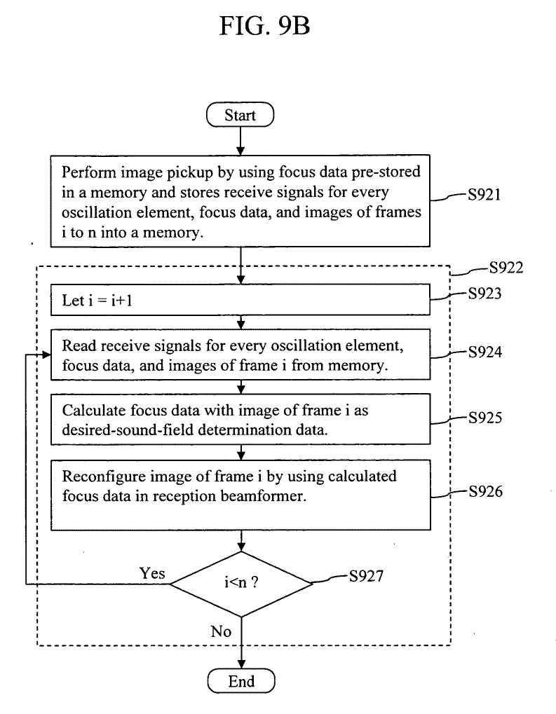

Next, a second embodiment of the present invention will be described with reference to FIGS. 9A and 9B. Although in the first embodiment, description has been made on the configuration in which focus data is calculated by real time processing with the image of i-th frame as desired-sound-field determination data, and is used in the focus data of one or both of the transmission beamformer and reception beamformer of the i+1 -th frame, the focus data calculated from the desired-sound-field determination data may be used only by the transmission beamformer 3, only by reception beamformer 5, or by both the transmission beamformer 3 and the reception beamformer 5. Moreover, in time wise, it may be reflected to image pickup in real time, or may be used off-line for image reconfiguration in non-real time. However, in the latter case in which off-line processing is performed, it is supposed that the memory 7 should store the focus data and the transmit and receive signals of the transmission beamformer 3 and the reception beamformer 5.

Examples of the processing other than the one described in the first embodiment are shown in FIGS. 9A and 9B. FIG. 9A shows a case in which focus data is calculated (S914′) by real time processing with the image of the i-th frame as the desired-sound-field determination data and the image is reconfigured (S915′) by using the reception beamformer in the same i-th frame. In this case, it is necessary to store receive signal of the frame i for every oscillation element and focus data in a memory (S913 and 913′). It is noted that S910 (S911 to S915) represents the processing of the first (0-th) frame, S910′ (S911′ to S915′) represents the processing of the subsequent frames, and corresponding processing are designated by primed numbers since there are many corresponding processing.

FIG. 9B represents the case in which focus data is calculated by off-line processing with the image of the i-th frame as the desired-sound-field determination data and the image is reconfigured by using the reception beamformer of the same i-th frame. In an off-line processing, calculated focus data can be used only for the reception beamformer. The step S921 represents the process of physically transmitting/receiving sound to and from an image pickup target, and the step S922 (S923 to S927) represents the process of image reconfiguration by off-line processing. In the off-line processing 5922, receive signals for each element, focus data, and images are read from the memory for every frame while updating the frame number i (S924), focus data is calculated (S925), and using the same, the image of the same i-th frame is reconfigured (S926).

According to the above described configuration, particularly to the one shown in FIG. 9A, although the frame rate is lower than that of the first embodiment, a high suppression effect against a fast-moving noise source can be achieved so that a high quality image with a high signal-to-noise ratio can be obtained. Further, according to the configuration as described above, particularly to the configuration shown in FIG. 9B, even when the computation capacity of the image pickup apparatus is insufficient, it is possible to suppress the effect of a noise source and obtain a high-quality image with a high signal-to-noise ratio.

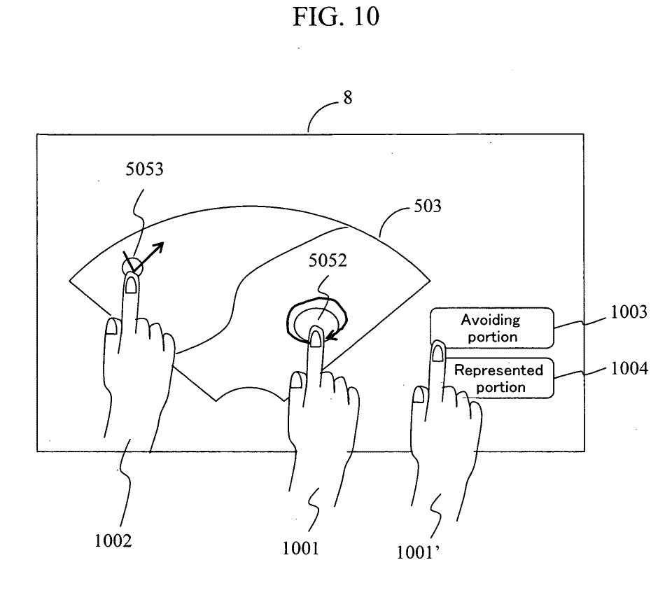

Next, a third embodiment of the present invention will be described with reference to FIG. 10.

FIG. 10 is a conceptual diagram to illustrate an example of the processing of the desired-beam-shape setting part 61 in the third embodiment of the present invention. Numeral 8 represents a display part, and 1001 and 1002 represent fingers of the image pickup operator showing an example of input means 9. That is, the present embodiment illustrates an example in which the input means is a touch-panel system having input button indications 1003 and 1004. On the display part 8, an image pickup result 503 is displayed as the desired-sound-field determination data. There are a strong reflector 5052 which acts as a noise source, and a site 5053 to be visualized in the image pickup result 503. The image pickup operator inputs one or more of an avoiding portion and a desired beam shape by a touch-panel system.

Describing an example of inputting an avoiding portion, for example, the image pickup operator views the image pickup result 503 displayed as the desired-sound-field determination data and visually recognizes a strong reflector 5052 as an avoiding portion, touches the position of the strong reflector 5052 on the touch-panel (1001), then touches an input button indication 1003 which indicates that an avoiding portion is specified (1001′). Then, an avoiding portion detection part 612 performs internal processing to calculate the position and signal intensity of a contact site to be for example 0 dB at a position of a deflection angle of 30±5 degrees, and provides them as the position and signal intensity of the avoiding portion. Further, arrangement may be made such that the image pickup operator views the image pickup result 503 displayed as the desired-sound-field determination data, visually recognizes and regards the site to be represented 5053 as a represented portion, touches the position of the represented portion 5053 on the touch-panel (1002), and then touches the input button indication 1004 which indicates that a represented portion is specified. In this case, the avoiding portion detection part 612 performs internal processing to calculate the position and signal intensity of a represented portion to be −50 dB at a position of a deflection angle of −25±2 degrees, and provides them as the position and signal intensity of the represented portion. It is noted that contact onto the touch panel may either be in point-wise manner as shown in the example of 5053 or in line-wise manner as shown in the example of 5052.

The avoiding portion detection part 612 is supposed to include a detection part for inputting a contact and outputting the range and signal intensity of a specified portion. For example, the above described detection part detects the position of contacted pixels on the touch-panel, and for example with the average of the contacted pixels being the center and twice the variance of the contacted pixels being a proximity distance, determines and detects that the site, which exhibits small change in the luminance of the desired-sound-field determination data, for example, has a variation of not more than 1/10 of the difference between a maximum luminance and a minimum luminance of the entire image 503, belongs to the same structure, and outputs a specified range, for example, a range of deflection angle of 30±5 degrees for the contact 1001 in the above described example, further outputting a representative luminance of the aforementioned site, for example, an average luminance, or a luminance obtained by subtracting an integer multiple of the variance of luminance from the average luminance as the signal intensity of specified part.

Next, the position and signal intensity of the avoiding portion and represented portion (respectively 0 dB at a position of a deflection angle of 30±5 degrees and −50 dB at a position of a deflection angle of −25±2 degrees) which are detected by the avoiding portion detection part 612 as described above are inputted to the desired-beam-shape converting part 613, which converts them into a desired beam shape. In the above described example, regardless of the direction of the main beam, it is supposed that the desired beam shapes are the directivities 5061 to 5064 (FIG. 5) each having a suppressed portion which is smaller than the main beam by −50 dB at a position of a deflection angle of 30±5 degrees.

It is noted that although an example of a touch-panel system is shown, which will not limit the input means. An example thereof may be one or more of utensils such as a pen, a keyboard, and a mouse.

According to the configuration as described above, it is possible to securely avoid the influence of noise sources which are known in advance, such as a therapeutic probe of HIFU. Further, it is possible to reflect the image pickup operator's intention more flexibly.

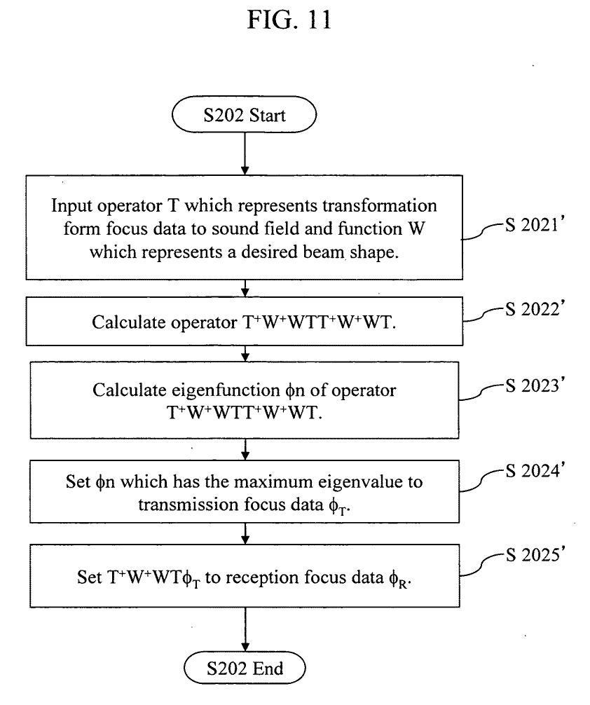

Next, a fourth embodiment of the present invention will be described with reference to FIG. 11.

FIG. 11 is a flowchart to illustrate an example of the processing (S202) of a focus data generation part 62 in the fourth embodiment of the present invention. Upon the start of the processing of the focus data generation part 62 (S202, START), an operator T which represents the transformation from focus data to sound field and a function W which represents a desired beam shape are inputted (S2021′), an operator T⁺W⁺WTT⁺W⁺WT is calculated (S2022′), the eigenfunction On of the operator T⁺W⁺WTT⁺W⁺WT is calculated (S2023′), φn which has a maximum eigenvalue is set to transmission focus data φ_{T} (S2024′), and T⁺W⁺WTφ_{T} is set to reception focus data φ_{R} (S2025′).

Using the equations described below, description will be made on the arrangement by which when it is desired to generate a beam having a shape W, transmission and reception focus data are determined as the eigenfunction φ of the operator T⁺W⁺WTT⁺W⁺WT and T⁺W⁺WTφ in FIG. 11A.

$$\begin{matrix}\{ \begin{matrix}{B}_{T} = T {φ}_{T} \\ {B}_{R} = T {φ}_{R}\end{matrix} & ( 5 ) \\ \begin{matrix}J = {( {WB}_{R} )}^{+} ( {WB}_{T} ) - {λ}_{T} {‖ {φ}_{T} ‖}^{2} - {λ}_{R} {‖ {φ}_{R} ‖}^{2} \\ = {( WT {φ}_{R} )}^{+} ( WT {φ}_{T} ) - {λ}_{T} {φ}_{T}^{+} {φ}_{T} - {λ}_{R} {φ}_{R}^{+} {φ}_{R} \\ = {φ}_{R}^{+} {T}^{+} {W}^{+} WT {φ}_{T} - {λ}_{T} {φ}_{T}^{+} {φ}_{T} - {λ}_{R} {φ}_{R}^{+} {φ}_{R}\end{matrix} & ( 6 ) \\ δ J = δ {φ}_{R}^{+} \cdot ( \begin{matrix}{T}^{+} {W}^{+} WT {φ}_{T} - \\ {λ}_{R} {φ}_{R}\end{matrix} ) + ( \begin{matrix}{φ}_{R}^{+} {T}^{+} {W}^{+} WT - \\ {λ}_{T} {φ}_{T}^{+}\end{matrix} ) δ {φ}_{T} & ( 7 ) \\ \begin{matrix}δ J = 0 \\ ⇔ \{ \begin{matrix}{T}^{+} {W}^{+} WT {φ}_{T} = {λ}_{R} {φ}_{R} \\ {T}^{+} {W}^{+} WT {φ}_{R} = {λ}_{T} {φ}_{T}\end{matrix} \\ ⇔ \{ \begin{matrix}( {T}^{+} {W}^{+} WT ) ( {T}^{+} {W}^{+} WT ) {φ}_{R} = \frac{{λ}_{R}}{{λ}_{T}} {φ}_{R} \\ {T}^{+} {W}^{+} WT {φ}_{R} = {λ}_{T} {φ}_{T}\end{matrix}\end{matrix} & ( 8 )\end{matrix}$$

The above described Equations (5) to (8) correspond to Equations (1) to (4) described in the first embodiment. Suppose that a beam is created from transmission focus data φ_{T} and reception focus data φ_{R}. In this case, since the operator representing the transformation from focus data to sound field is represented by T, the shape of the transmission beam is given as B_{T} and the shape of the reception beam is given as B_{R} as shown in Equation (5). Here, the degree of coincidence between the transmission/reception beam shapes B_{R} and B_{T} created by focus data φ_{T} and φ_{R} and the desired beam shape W is represented by the product of respective beam shapes and the desired shape, the product of WB_{R} and WB_{T}. On the other hand, the total of the transmission sound pressure outputted from the probe is represented by the square of norm ∥φ_{T}∥² of transmission focus data φ_{T} and the total of the received sound pressure is represented by the square of norm ∥φ_{R}∥² of reception focus data φ_{R}.

Here, suppose that it is a goal to determine focus data for creating a beam which coincides with a desired beam shape W as much as possible under the condition of constant acoustic intensity at a focus point. Then, formulating this problem by means of the calculus of variations, the first line of Equation (6) is obtained as the evaluation function. λ_{T} and λ_{R} are undetermined coefficients of Lagrange, and the term to which the coefficient is applied is a constraint. By substituting Equation (5) into the first line of the evaluation function (6) and rearranging it, the first line of the Equation (5) is rearranged as the third line of Equation (6), and by taking variations thereof, Equation (7) will result. Since minute changes δφ_{T}⁺ δφ_{R}⁺ are arbitrary, it is seen that by deforming the first line of conditional equation (8) in which variations is zero, the problem ends up to simultaneous equations with two variables, which is an eigenvalue problem obtained at the third line of equation (8), each of which relates to φ_{T} and φ_{R}. That is, determining the eigenfunction φ of the operator T⁺W⁺WTT⁺W⁺WT for reception or transmission focus data and using T⁺W⁺WTφ as transmission or reception focus data will result in a generation of a beam which has a shape as close as possible to the shape W under the condition of a constant acoustic intensity at a focus point.

Here, although one eigenvalue equation is obtained in Equation (4) of the first embodiment, Equation (8) in the fourth embodiment is different in that simultaneous equations with two variables are obtained. Since that is simultaneous equations with two variables, it is possible to determine different focus data for transmission and reception.

According to the configuration as described above, transmission focus data and reception focus data which is different from the transmission focus data are determined respectively, and it is possible to form a beam in which the directivity in transmission and reception is suppressed not only in side lobes but also in grating lobes. As a result of that, a high-quality image pickup becomes possible even when a deflection angle is increased at a sector probe, enabling to set a wide image pickup range. Moreover, when scanning is performed with a large oblique angle in a linear probe, for example, having a wide image pickup range of trapezoidal shape, or even when compound processing is performed, a high-quality image pickup is enabled. Especially in the case of compound processing, since the number of summing frames increases, it is possible to achieve not only a direct effect of improving signal-to-noise ratio per one scan but also an indirect effect of improving contrast because of the increase in the number of summing frames. Further, it becomes possible to reduce the influence of a grating which is inevitably produced as a result of the pitch between elements being not less than the half of the wavelength in a high-frequency probe and a 2-dimensional probe, and thus a high-quality image pickup is made possible.

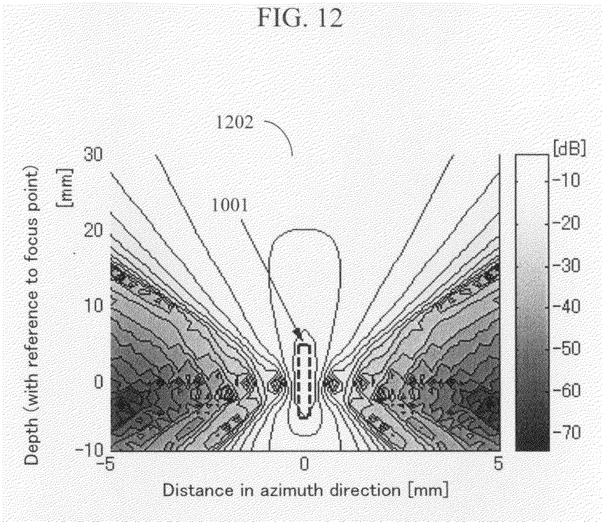

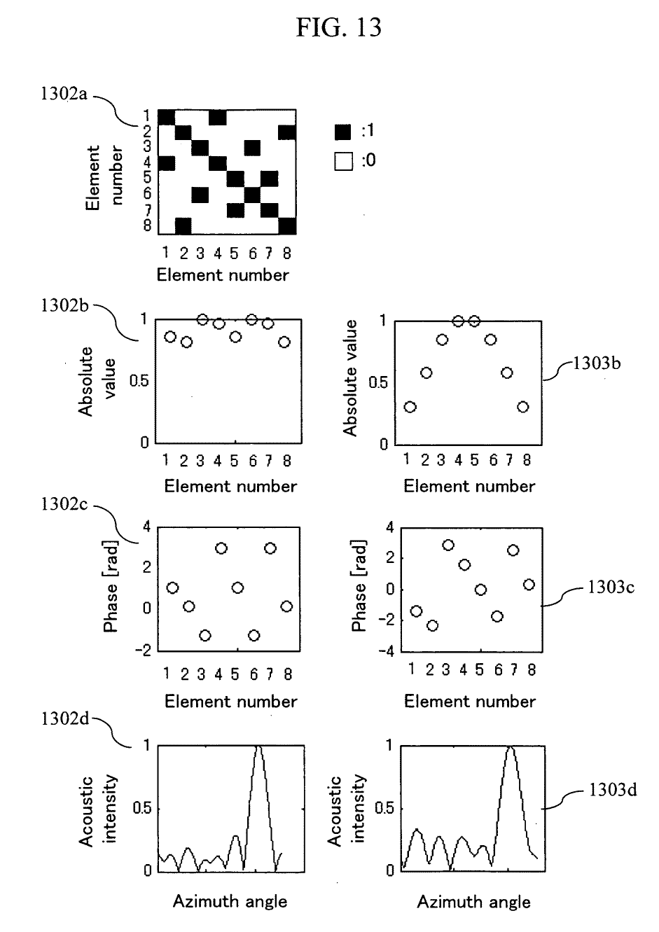

Hereafter, with reference to FIGS. 12 and 13, a fifth embodiment of the present invention will be described. Hereafter, unless otherwise stated, description will be made based on an example in which the operator is discrete, φ is spatially one-dimensional and temporally zero-dimensional, T is spatially one-dimensional and temporally zero-dimensional, and W is spatially one dimensional and temporally zero-dimensional.

The ultrasonic apparatus of the present embodiment includes a memory for storing information, and the memory includes a receive signal storage part for storing receive signals for each of the plurality of elements. The data acquired at the desired-sound-field determination data acquisition part is the receive signals for each of the plurality of elements stored in the receive signal storage part, an image generation part reads receive signals for each of the plurality of elements from the receive signal storage part to reconfigure an image using the focus data outputted by a focus data generation part. The focus data generation part outputs focus data proportional to eigenfunction φ of the operator T⁻¹W⁻¹ WT with T as the operator representing the transformation from focus data to sound field and with W as the function representing the desired beam shape, or outputs the transmission focus data proportional to the eigenfunction φ_{T} of the operator T⁺W⁺WTT⁺W⁺WT and the reception focus data φ_{T} proportional to T⁺W⁺WTφ_{T}.

In the processing of the focus data generation part, φ may be either spatially one-dimensional or two-dimensional. When the spatial dimension of φ is one-dimensional, it corresponds to a one-dimensional probe, and T will be a two-dimensional matrix and W will be a two-dimensional matrix. When the spatial dimension of φ is two-dimensional, it corresponds to a two-dimensional probe, and T will be a 3rd order tensor and W will be a two-dimensional matrix. According to the configuration as described above, it is possible to determine focus data either when the probe is one-dimensional or when two-dimensional.

Alternatively, in the processing of the focus data generation part, the frequency-space dimension or temporal dimension of φ may be zero-dimensional or one-dimensional. When the frequency-space dimension or temporal dimension of φ is zero-dimensional, T will be a two-dimensional matrix, and W will be a two-dimensional matrix. When the frequency-space dimension or temporal dimension of φ is one-dimensional, T will be a third-order tensor and W will be a two-dimensional matrix. According to the configuration as described above, when the frequency-space dimension or temporal dimension is zero-dimension, the focus data of the probe can be determined. When the frequency-space dimension or temporal dimension is one-dimensional, it is not only possible to determine focus data of the probe but also possible to design an optimum pulse for every element.

Alternatively, in the processing of the focus data generation part, the spatial dimension of W may be any of one-dimensional, two-dimensional, and three-dimensional. When the spatial dimension of W is one-dimensional, T will be a two-dimensional matrix, and W will be a two-dimensional matrix. When the spatial dimension of W is two-dimensional, T will be a third-order tensor, and W will be a fourth-order tensor. When the spatial dimension of W is three-dimensional, T will be a fourth-order tensor, and W will be a sixth-order tensor.

FIG. 12 illustrates a sound field formed from a desired beam shape and focus data calculated by the focus data generation part when the spatial dimension of W is two-dimensional. Numeral 1001 represents the desired beam shape, and numeral 1202 represents the sound field formed. It is seen that there is obtained a sound field which coincides well with the desired beam shape 1001, and the length of which in the depth direction is several times longer than usual. When such a long focusing region is obtained, a high-quality image is obtained in a wide depth region. Further, it is possible to uniformly break a contrast agent. Further, it is possible to generate a stronger high-frequency component than before at a deeper depth than before so that an image which can combine the penetration with the image quality in deep part can be obtained.

According to the configuration as described above, it is possible to perform any of beam designs in one-dimensional space, in two-dimensional space, and in three-dimensional space.

Alternatively, in the processing of the focus data generation part, the dimension of frequency space or temporal dimension of W may either be zero-dimensional or one-dimensional. When the dimension of frequency space or temporal dimension of W is zero-dimensional, T will be a two-dimensional matrix, and W will be a two-dimensional matrix. When the dimension of frequency space or temporal dimension of W is one-dimensional, T will be a third-order tensor and W will be a fourth-order tensor.

According to the configuration as described above, it becomes possible to perform a beam design in association with time.

Alternatively, in the processing of the focus data generation part, T may include a discretizing operation G (T′=TG). In this case, T′ will be a two-dimensional matrix and W will be a two-dimensional matrix.

FIG. 13 illustrates a connection pattern and focus data calculated by the focus data generation part, and a sound field formed thereby when two elements of a one-dimensional probe made up of 8 elements to create a main beam in the direction of a deflection angle of 45 degrees.

The following equation is an example of a discretizing operator G.

$$G = \begin{matrix}1 \\ 2 \\ 3 \\ ⋮ \\ j \\ ⋮ \\ \end{matrix} {[ \begin{matrix}1 & 1 & & & & & \\ 1 & 1 & & & & & \\ & & 1 & … & 1 & & \\ & & ⋮ & & ⋮ & & \\ & & 1 & … & 1 & & \\ & & & & & ⋱ & \\ & & & & & & \end{matrix} ]}^{\begin{matrix}1 & 2 & 3 & … & j & … & \end{matrix}} \times \frac{1}{2}$$

The discretizing operator G is a two-dimensional matrix with rows (number of elements) and columns (number of elements), that is 8 rows and 8 columns, and when considering a case in which the elements are numbered from 1 to 8, and element 1 and element 2, and element 3 and element j are connected, the matrix will satisfy the following equations.

G₁₁=G₁₂=G₂₁=G₂₂=1/2,

G₃₃=G₃ⱼ=Gⱼ₃=Gⱼⱼ=1/2,

The focus data generation part calculates the operator G for all the possible connection patterns, that is, ₈C₂·₆C₂·₄C₂·₂C₂ 2 connection patterns, calculates T′=TG for each G, calculates the operator T′⁺W⁺WT′, calculates the eigenfunction φn of the operator T′⁺W⁺WT′, and sets φn, which has the maximum eigenvalue among the eigenfunctions for all the G, to the focus data.

The matrix G representing the connection pattern, which has been thus outputted, is 1302a, the absolute value of the focus data is 1302b, the phase of the focus data is 1302c, and the sound field formed by those connection pattern 1302a and focus data 1302b and 1302c is 1302d. For comparison, the absolute value of the focus data, which is outputted by the focus data generation part when there is no connection, is shown by 1303b, the phase of the focus data is shown by 1303c, and the sound field is shown by 1303d, which is formed when elements having close values of the phase of the focus data are connected in a simple manner without numerically optimizing them as in the present invention. It is noted that the horizontal axes of graphs 1302a, 1302b, 1302c, 1303b, and 1303c and the vertical axis of 1302a represent element numbers, and the horizontal axes of the graphs 1302d and 1303d represent azimuth angles, and the vertical axes represent acoustic intensities (linear indication).

Since the intensity of the focus data is larger in the case of the present invention (1302b) than in a convention example (1303b) based on a simple idea, the sound pressure is ensured and images with good penetration are obtained. Moreover, in the sound field formed, side lobes are more suppressed in the present invention (1102d) than in the conventional example (1303d), and an image of better signal-to-noise ratio has been obtained.

According to the configuration as described above, for example, when the number of elements is far more than the number of signal lines and a plurality of elements need to be connected to one signal line, it is possible to optimize not only focus data but also the pattern of the grouping of elements for a desired beam.

### DESCRIPTION OF REFERENCE NUMERALS

1 probe
2 apparatus main body
3 transmission beamformer
4 amplification means
5 reception beamformer
6 signal processing part
7 memory
8 display means
9 input means
10 controller
61 target-setting-data acquisition part
62 avoiding portion detection part
63 desired-beam-shape setting part
64 focus data generation part

1. An ultrasonic apparatus, comprising:
a probe including a plurality of elements for transmitting or receiving ultrasound;
a transmission beamformer for imparting directivity to an ultrasonic signal upon transmission to a subject by said plurality of elements;
a reception beamformer for summing each ultrasonic signal received by said plurality of elements, along with directivity thereof;
a signal processing part for signal-processing and imaging the signal outputted by said reception beamformer; and
display means for displaying the image outputted by said signal processing part, wherein
said signal processing part includes a desired-beam-shape setting part for setting a desired beam shape, a focus data generation part which receives said desired beam shape as input and calculates focus data to generate a beam along said desired beam shape, and an image generation part for generating an image.

2. The ultrasonic apparatus according to claim 1, wherein
said desired-beam-shape setting part comprises: a desired-sound-field determination data acquisition part for acquiring data to set a desired sound field; an avoiding portion detection part for detecting a position and intensity of a site to be avoided from said desired-sound-field determination data; and a desired-beam-shape converting part for converting said position and intensity of the site to be avoided into the desired beam shape; and
at least one of said transmission beamformer and said reception beamformer generates a beam by using the focus data outputted by said focus data generation part.

3. The ultrasonic apparatus according to claim 1, further comprising
a memory for storing information, wherein said memory comprises a receive signal storage part for storing receive signals for each of said plurality of elements,
said desired-beam-shape setting part comprises a desired-sound-field determination data acquisition part for acquiring data to set a desired sound field, an avoiding portion detection part for detecting a position and intensity of a site to be avoided from said desired-sound-field determination data, and a desired-beam-shape converting part for converting said position and intensity of the site to be avoided into the desired beam shape,
the data for setting said desired sound field is receive signals for each of said plurality of elements stored in said receive signal storage part, and
said image generation part reads said receive signals for each of said plurality of elements from said receive signal storage part and reconfigures an image by using focus data outputted by said focus data generation part.

4. The ultrasonic apparatus according to claim 1, wherein
with T being an operator which represents the transformation from focus data to sound field and with W being a function to represent said desired beam shape, said focus data generation part outputs focus data proportional to an eigenfunction φ of an operator T⁻¹W⁻¹WT.

5. The ultrasonic apparatus according to claim 2, wherein
with T being an operator which represents the transformation from focus data to sound field and with W being a function to represent said desired beam shape, said focus data generation part outputs focus data proportional to an eigenfunction φ of an operator T⁻¹W⁻¹WT.

6. The ultrasonic apparatus according to claim 3, wherein
with T being an operator which represents the transformation from focus data to sound field and with W being a function to represent said desired beam shape, said focus data generation part outputs focus data proportional to an eigenfunction φ of an operator T⁻¹W⁻¹WT.

7. The ultrasonic apparatus according to claim 1, wherein
with T being an operator which represents the transformation from focus data to sound field and with W being a function to represent said desired beam shape, said focus data generation part outputs transmission focus data proportional to an eigenfunction φ_{T} of an operator T⁺W⁺WTT⁺W⁺WT and reception focus data φ_{T} proportional to T⁺W⁺WTφ_{T}.

8. The ultrasonic apparatus according to claim 2, wherein
with T being an operator which represents the transformation from focus data to sound field and with W being a function to represent said desired beam shape, said focus data generation part outputs transmission focus data proportional to an eigenfunction φ_{T} of an operator T⁺W⁺WTT⁺W⁺WT and reception focus data φ_{T} proportional to T⁺W⁺WTφ_{T}.

9. The ultrasonic apparatus according to claim 3, wherein
with T being an operator which represents the transformation from focus data to sound field and with W being a function to represent said desired beam shape, said focus data generation part outputs transmission focus data proportional to an eigenfunction φ_{T} of an operator T⁺W⁺WTT⁺W⁺WT and reception focus data φ_{T} proportional to T⁺W⁺WTφ_{T}.

10. The ultrasonic apparatus according to claim 4, wherein
said display means displays at least one of said desired-sound-field determination data, said desired beam shape, and said focus data, and
said ultrasonic apparatus includes input means for inputting at least one of said desired beam shape and said focus data through an operation by an image pickup operator.

11. The ultrasonic apparatus according to claim 5, wherein
said display means displays at least one of said desired-sound-field determination data, said desired beam shape, and said focus data, and
said ultrasonic apparatus includes input means for inputting at least one of said desired beam shape and said focus data through an operation of by an image pickup operator.

12. The ultrasonic apparatus according to claim 6, wherein
said display means displays at least one of said desired-sound-field determination data, said desired beam shape, and said focus data, and
said ultrasonic apparatus includes input means for inputting at least one of said desired beam shape and said focus data through an operation of by an image pickup operator.

13. The ultrasonic apparatus according to claim 7, wherein
said display means displays at least one of said desired-sound-field determination data, said desired beam shape, and said focus data, and
said ultrasonic apparatus includes input means for inputting at least one of said desired beam shape and said focus data through an operation of by an image pickup operator.

14. The ultrasonic apparatus according to claim 8, wherein
said display means displays at least one of said desired-sound-field determination data, said desired beam shape, and said focus data, and
said ultrasonic apparatus includes input means for inputting at least one of said desired beam shape and said focus data through an operation of by an image pickup operator.

15. The ultrasonic apparatus according to claim 9, wherein
said display means displays at least one of said desired-sound-field determination data, said desired beam shape, and said focus data, and
said ultrasonic apparatus includes input means for inputting at least one of said desired beam shape and said focus data through an operation of by an image pickup operator.
